(19) United States
(12) Patent Application Publication
(10) Pub. No.: US 2010/0055073 A1
(43) Pub. Date: Mar. 4, 2010

(54) TREATMENT OF PERVASIVE DEVELOPMENTAL DISORDERS

(76) Inventors: Andrew Sean Lynch, Liverpool (GB); Kerry Louisa Hambleton, Northwich (GB); Stephen Leyland Smith, Liverpool (GB)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/483,580

(22) Filed: Jun. 12, 2009

Related U.S. Application Data
(63) Continuation of application No. PCT/GB2007/004815, filed on Dec. 14, 2007.

(30) Foreign Application Priority Data
Dec. 14, 2006 (GB) 0624879.3

(51) Int. Cl. A61K 31/405; A61K 35/74; A61P 25/00

(52) U.S. Cl. 424/93.4; 514/419

(57) ABSTRACT

An orally administrable nutritional formulation for use in the treatment of Pervasive Developmental Disorders (e.g. Autism, Asperger disorder or Retts disorder) contains free amino acids as the sole source of protein. The formulation may be administered to the patient as the sole daily source of protein or as a supplemental source of protein

### CROSS-REFERENCE TO RELATED APPLICATIONS

This application is a continuation of International Application No. PCT/GB2007/004815 filed Dec. 14, 2007, which claims the benefit of British Application No. 0624879.3 filed Dec. 1, 2006.

The present invention relates to the treatment of Pervasive Developmental Disorders, e.g. Autism.

Autism is a complex developmental disease characterised by impairments in social functioning, communication and flexibility of thought and behaviour (Wing 96). Impairments become apparent in early childhood (˜2yo) and last into adulthood, having a severe effect on an individual's learning and social integration.

Estimates of prevalence have increased markedly in recent years, probably due to improved diagnostic procedures and increased public and clinical awareness of the condition (Wing 02): current best estimates range from 1:1000 to 1:200 (Fombonne 99).

Autism is a member of an extended family of Pervasive Developmental Disorders (PDD), which also includes Asperger and Rett disorders. All PDDs require the triad of social, communication and behavioural impairments identified by Wing for diagnosis. Similar disorders, such as Attention Deficit Disorder (ADD), hyperactivity disorders and schizophrenia, generally exhibit such impairments, but their presence is not required for diagnosis. The group of disorders commonly presenting with Wing's triad, whether required for diagnosis or not, is often referred to as the ‘autistic spectrum’.

Disease aetiology is unknown, with the consensus expert opinion being that a genetic predisposition may interact with multivariate environmental factors to cause the disorder. The uncertainty over possible causes and disease mechanisms has resulted in the poor therapeutic options available for affected individuals.

Current pharmaceutical treatments generally address secondary symptoms of the disease, not the core aspects. For example, neuroleptics are commonly used to moderate aggression and biting behaviour, SSRIs to treat depression, and stimulants to improve impulse control or hyperactivity. Such approaches are acknowledged to be of restricted worth in control of the condition generally (Bostic 05).

Opioid peptides in the urine of autistic children were first observed in the late 1980s (Israngkun 86). Such peptides are formed by processing of dietary gluten and casein proteins in the gut, and are normally not absorbed into the body. Gluten forms gliadomorphins and casein forms casomorphins. These peptides, collectively termed exorphins (Zioudrou 79), are of essentially identical structure and are potent psychosis-inducing factors (Lindstrom 84).

If gut structure is compromised, these peptides can enter the body. The ‘leaky gut’ or ‘opioid excess’ hypothesis was developed proposing that autism, at least in a subset of the population, may be caused by entry of exorphins into the body from a leaky gut, allowing transport in the blood to the brain, and subsequent damage to the brain.

The hypothesis has drawn support over the years from further characterisation of the urinary peptides (Shattock 90, Reichelt 91), definitive demonstrations of leaky gut in autistics (e.g., D'Eufemia 96), as well as structural characterisation of the GI pathology, which has been described as ‘a subtle new variant of inflammatory bowel disease that lacks the specific features of Crohn's disease or ulcerative colitis’. Multiple studies have confirmed a non-specific colitis along the length of the bowel, along with specific inflammatory lesions in lymph nodes of the ileum and colon (Wakefield 98, Sabra 98, Horvath 99, Wakefield 00).

GI problems associated with autism have been noted for many years and are estimated to affect approx. 20-30% of the population. Symptoms range from bloating and abdominal pain to loose stools and diarrhoea, and correlations with food intolerances, principally to milk and wheat, have been noted (e.g., Goodwin 71, Lightdale 01). Interestingly, urinal peptides are present in >90% of autistics, suggesting there may be a sub-clinical GI dysfunction in most autistics, which is only expressed as significant GI problems in the 20-30% noted above.

The original observations of urinary peptides and the leaky gut/opioid excess hypothesis led to the first clinical studies of dietary interventions, specifically casein- and gluten-free diets (Reichelt 90, Knivsberg 95, Lucarelli 95). Unfortunately, although these studies did suggest that dietary intervention could improve both GI and autistic measures over long time periods (up to 4 years), their design did not allow a direct effect between dietary control and clinical efficacy to be confirmed.

There are ongoing studies to confirm the efficacy of exclusion diet (e.g., NIMH 04), but one recent published study has achieved the methodological quality required to support clinical effect (Knivsberg 02). The study compared the impact of a casein/gluten-free diet versus normal diet on clinical autistic measures over 1 year in 20 autistic children with abnormally high urinary peptide levels. Significant reduction in autistic traits was observed in those on restricted diet. In addition to anecdotal reports from parents, case-studies and uncontrolled studies, this study lends ‘tentative support’ to dietary intervention as an effective option in autism (Millward 05).

The available data suggests that a sub-clinical GI impairment exists in the majority of autistics which is expressed as full-blown GI symptoms in a significant subset of the population. Exclusion diets have been demonstrated to improve both GI and autistic function and to maintain the improvement for long time periods.

According to a first aspect of the present invention there is provided the use of an orally administrable nutritional formulation containing free amino acids as the sole source of protein for the manufacture of a medicament for administration as the sole daily source of protein for the treatment of Pervasive Developmental Disorders.

According to a second aspect of the present invention there is provided a method of treating a Pervasive Developmental Disorder comprising orally administering to a person in need of such treatment an effect amount of a nutritional formulation containing free amino acids as the sole source of protein, said amino acids in the nutritional formulation being the sole daily source of protein.

We have found, and this forms the basis of the present invention, that oral administration of a nutritional formulation containing free amino acids as the sole protein source in the formulation (so that the nutritional formulation is devoid of protein and/or protein fragments) in conjunction with the nutritional formulation providing the only protein source that is administered to the patient is an effective method for the treatment of Pervasive Developmental Disorders. In other words, the amino acids in the nutritional formulation are the sole source of protein in the patient's diet. Thus the patient receives no whole protein or protein fragments and there is therefore no risk of the production of exorphins such as gliadomorphins and casomorphins.

The invention is applicable particularly to the treatment of Autism but also has applicability to other Pervasive Developmental Disorders such as Asperger and Rett Disorders.

The nutritional formulation may be administered as the sole source of protein (i.e. as provided by the amino acids) for a limited period of time before dietary protein is reintroduced. This limited period of time may, for example, be at least one week, more generally at least two weeks. Typically the period would be 2-4 weeks or even longer depending on the circumstances. Reintroduction of dietary protein may follow an algorithm designed to detect which protein(s) is/are involved in individual patients.

The free amino acids present in the formulation may comprise L-alanine, L-arginine, L-aspartic acid, L-cystine, glycine, L-hitidine, L-isoleucine, L-lysine, L=methiunine, L-phenylalaine, L-proline, L-serine, L-threanine, L-tryptopahn, L-tyrosine, L-valine, L-carntine and taurine. The nutritional formulation may further comprise L-glutamine as a free amino acid. The presence or absence of glutamine will generally be dictated by the manner in which the nutritional formulation is produced. If heat treatment is required (e.g. for the production of a nutritional formulation in the form of a pasteurised drink) then the use of glutamine will generally be avoided to prevent “off-flavours”.

The nutritional formulation may contain at least one (and preferably all) of a carbohydrate, fat, vitamins, minerals and trace elements.

The formulation may be nutritionally complete.

Particularly suitable formulations for administration in accordance with the invention are Neocate and Paediatric E028 liquid, both available from SHS International Limited.

Although the invention is intended primarily for the treatment of Pervasive Developmental Disorders, we also envisage that it has application to the diagnosis of such conditions. Thus the nutritional formulation may be administered to a person suspected of suffering from a Pervasive Developmental Disorder to see whether there is any improvement in the condition. If so, this would at least assist in confirming a diagnosis of the condition in that person.

### REFERENCES

Bostic 2005, Expert Opin Emerg Drugs 10: 521.

D'Eufemia 1996, Acta Paediatr. 85:1076

Fombonne 1999, Psychol. Med. 29:769

Goodwin 1971, J. Autism Child Schiz. 1:48

Harapocos 1975, DIPAB Herning, Norway

Horvath 1999, J. Pediatr. 135:559

Israngkun 1986, Neurochem. Pathol. 5:51

Knivsberg 1995, Scand. J. Educ. Res. 39:223

Knivsberg 2002, Nutr. Neurosci. 5:251

Liacouras 2003, Am. J Gastroenterol. 98:777

Lightdale 2001, Clin. Perspect. Gastroenterol. 1:56

Lindstrom 1984, Am. J. Psych. 41:1059

Lucarelli 1995, Panminerva Med. 37:137

Millward 2005, Cochrane Database Syst. Rev. CD003498

NIMH 2004, http://clinicaltrials.gov/show/NCT00090428

Rainford 2005, Internal SHS report

Reichelt 1990, J. Appl. Nutr. 42:1

Reichelt 1991, Brain Dysfunct. 4:308

Sabra 1998, Lancet 352:234

Shattock 1990, Brain Dysfunct. 3:328

Wakefield 1998, Lancet 35:637

Wakefield 2000, Am. J. Gastroenterol. 95:2285

Wing 1996, BMJ312:327

Wing 2002, Mental Retardation Dev. Disabilities Res. Dev. 8:151

Zioudrou 1979, J. Biol. Chem. 254:2446

1. Use of an orally administrable nutritional formulation comprising amino acids as the sole protein source, fat and carbohydrates wherein
a. the amino acid composition comprises at least L-alanine, L-arginine, L-aspartic acid, L-cystine, glycine, L-histidine, L-isoleucine, L-lysine, L-methionine, L-phenylalanine, L-proline, L-serine, L-threonine, L-tryptophan, L-tyrosine, L-valine, L-camitine and taurine;
b. the fat comprises at least 10 wt % long chain polyunsaturated fatty acids based on the weight of the fat in the composition;
c. the carbohydrates comprising between 5 and 50 wt % dietary fibres based on the total weight of the carbohydrates in the composition; and
wherein the weight percentage of amino acids is at least 5%, of fat is between 0-20% and the carbohydrates at between 10 and 70% of the dry weight of the composition,
for the manufacture of a medicament for administration as the sole daily source of protein or as a supplemental source of protein for the treatment of Pervasive Developmental Disorders.

2. The use as claimed in claim 1 wherein the Pervasive Developmental Disorder is autism.

3. The use as claimed in claim 1 wherein the Pervasive Developmental Disorder is Asperger disorder or Retts disorder.

4. The use as claimed in claim 1 wherein the free amino acids additionally comprise L-glutamine.

5. The use as claimed in claim 1 wherein the weight percentage based on total dry weight of the composition of amino acids is between 10 and 15 wt %, fat is between 20 and 25 wt % and carbohydrate is between 40 and 65 wt %.

6. The use as claimed in claim 1 wherein the nutritional formulation incorporates vitamins and minerals.

7. The use as claimed in claim 1 wherein the formulation incorporates probiotic bacteria.

8. Nutritional formulation comprising amino acids as the sole protein source, fat and carbohydrates wherein
a. the amino acid composition comprises at least L-alanine, L-arginine, L-aspartic acid, L-cystine, glycine, L-histidine, L-isoleucine, L-lysine, L-methionine, L-phenylalanine, L-proline, L-serine, L-threonine, L-tryptophan, L-tyrosine, L-valine, L-carnitine and taurine;
b. the fat comprises at least 10 wt % long chain polyunsaturated fatty acids based on the weight of the fat in the composition;
c. the carbohydrates comprising between 5 and 50 wt % dietary fibres based on the total weight of the carbohydrates in the composition; and
wherein the weight percentage of amino acids is at least 5%, of fat is between 0-20% and the carbohydrates at between 10 and 70% of the dry weight of the composition,
for the treatment of Pervasive Developmental Disorders.

9. A formulation as claimed in claim 8 wherein the Pervasive Developmental Disorder is autism.

10. A formulation as claimed in claim 8 wherein the Pervasive Developmental Disorder is Asperger disorder or Retts disorder.

11. A formulation as claimed in claim 8 wherein the free amino acids additionally comprise L-glutamine.

12. A formulation as claimed in claim 8 wherein the weight percentage based on total dry weight of the composition of amino acids is between 10 and 15 wt %, fat is between 20 and 25 wt % and carbohydrate is between 40 and 65 wt %.

13. A formulation as claimed in claim 8 wherein the nutritional formulation incorporates vitamins and minerals.

14. A formulation as claimed in claim 8 wherein the formulation incorporates probiotic bacteria.

15. A method of treating a Pervasive Developmental Disorder comprising orally administering to a person in need of such treatment an effective amount of a nutritional formulation comprising amino acids as the sole protein source, fat and carbohydrates wherein
a. the amino acid composition comprises at least L-alanine, L-arginine, L-aspartic acid, L-cystine, glycine, L-histidine, L-isoleucine, L-lysine, L-methionine, L-phenylalanine, L-proline, L-serine, L-threonine, L-tryptophan, L-tyrosine, L-valine, L-carnitine and taurine;
b. the fat comprises at least 10 wt % long chain polyunsaturated fatty acids based on the weight of the fat in the composition;
c. the carbohydrates comprising between 5 and 50 wt % dietary fibres based on the total weight of the carbohydrates in the composition; and
wherein the weight percentage of amino acids is at least 5%, of fat is between 0-20% and the carbohydrates at between 10 and 70% of the dry weight of the composition,
said amino acids in the nutritional formulation being the sole daily source of protein or as a supplemental source of protein.

16. A method according to claim 15 wherein the nutritional formulation is supplemental to the diet.

17. A method as claimed in claim 15 wherein the nutritional formulation is the sole daily source of protein.

18. Composition comprising amino acids as the sole protein source, fat and carbohydrates wherein
a. the amino acid composition comprises at least L-alanine, L-arginine, L-aspartic acid, L-cystine, glycine, L-histidine, L-isoleucine, L-lysine, L-methionine, L-phenylalanine, L-proline, L-serine, L-threonine, L-tryptophan, L-tyrosine, L-valine, L-carnitine and taurine;
b. the fat comprises at least 10 wt % long chain polyunsaturated fatty acids based on the weight of the fat in the composition;
c. the carbohydrates comprising between 5 and 50 wt % dietary fibres based on the total weight of the carbohydrates in the composition; and
wherein the weight percentage of amino acids is at least 5%, of fat is between 0-20% and the carbohydrates at between 10 and 70% of the dry weight of the composition.

19. Composition according to claim 18 wherein the weight percentage based on total dry weight of the composition of amino acids is between 10 and 15 wt %, fat is between 20 and 25 wt % and carbohydrate is between 40 and 65 wt %.

20. A composition according to claim 18 further comprising probiotic bacteria.
